(19) United States
(12) Patent Application Publication
(10) Pub. No.: US 2012/0202778 A1
(43) Pub. Date: Aug. 9, 2012

(54) USE OF AT LEAST ONE OMEGA-3 FATTY ACID AND OF AT LEAST ONE POLYPHENOL FOR THE ENDOGENOUS SYNTHESIS OF EICOSAPENTANOIC ACID AND DOCOSAHEXANOIC ACID

(76) Inventors: Michel De Lorgeril, Meylan (FR); Patricia Salen, Meylan (FR)

(21) Appl. No.: 13/383,290

(22) PCT Filed: Jul. 15, 2010
(86) PCT No.: PCT/FR2010/051486

§ 371 Date: Apr. 26, 2012

(30) Foreign Application Priority Data
Jul. 15, 2009 (FR) 0954900

(51) Int. Cl. A61K 31/353; A23L 1/30; A23L 1/302; A23L 1/214; A23C 19/00; A23C 9/123; A23L 1/10; A61K 31/60; A23D 9/007

(52) U.S. Cl. 514/163; 514/456; 426/648; 426/311; 426/601; 426/607; 426/582; 426/583; 426/586; 426/549; 426/620; 426/615; 426/631

(57) ABSTRACT

The present invention relates to the use of at least one fatty acid of the omega-3 group, selected from among α-linolenic acid and stearidonic acid, and of at least one polyphenol as an agent for increasing the endogenous synthesis of eicosapentanoic acid and docosahexanoic acid, for preparing a food, health, or pharmaceutical composition to be administered to a human being or to an animal.

The present invention generally relates to animal food, notably human food and in particular to the complementation of this food with a composition containing a mixture of at least one omega-3 fatty acid with a polyphenol intended to increase the endogenous synthesis of omega-3 fatty acids with 20 or 22 carbon atoms (called very long chain (VLC) omega-3 fatty acids).

The main fatty acids of the omega-3 group are α-linolenic or ω3α acid (18:3; ALA), eicosapentaenoic acid (20:5;EPA) and docosahexaenoic acid (22:6;DHA). They are described as essential fatty acids because the animal, notably human, body does not know how to synthesize them or knows how to synthesize them in an insufficient amount. A minimum and regular supplement by food is considered as necessary by experts.

Large amounts of VLC fatty acids, notably EPA and DHA, are found in fatty fish. Nutritionists therefore recommend regular consumption of fish. Nevertheless, the increase in the consumption of fish world wide leads to overfishing and desertification of the seas. An alternative to overfishing lies in the existence of fish bred in marine farms. Plankton which contains substantial amounts of DHA and EPA, is indispensable for feeding farmed fish. The plankton is collected by harvesting boats which leads to plankton-depletion of the seas. This chain of events leads to an ecological upheaval and to violent conflicts with the fish industry.

ALA is found in plants, but this ALA is not efficiently transformed into the VLC fatty acids by animals, notably by humans. Therefore the use of ALA according to experts, cannot supplement a nutritional deficiency in VLC omega-3 of marine origin.

The present invention proposes a solution for replacing food consumption of VLC fatty acids of essentially marine origin.

The inventors realized that simultaneous provision of ALA and of polyphenols led to an increase in the endogenous synthesis of VLC fatty acids, notably, EPA and DHA.

It should be noted that a certain number of scientific articles indicate the positive effect of wine consumption on the plasma concentration of VLCs. Notably the article of De Lorgeril et al., (American Heart Journal, 2008, 155:175-181) studies the effect of wine on the metabolism of omega-3s and comes to the conclusion that an increase in the consumption of ALA associated with moderate consumption of ethanol represents an alternative to fish consumption. Also, the article of Guiraud et al. (British Journal of Nutrition 2008, 100, 1237-1244) studies the effect of ethanol on the metabolism of essential fatty acids in rats. The article of Di Giuseppe et al. (Am. J. Clin. Nutr. 2009, 89 :1-9) studies the concentrations of omega-3 fatty acids in wine consumers as compared with non-drinkers and drinkers of other alcoholic beverages and concludes that components of wine other than ethanol may have an effect on the plasma concentration of omega-3 fatty acids.

The present invention relates to the use of at least one fatty acid of the omega-3 group selected from α-linolenic acid (ALA) and stearidonic acid (SA) and of at least one polyphenol, as an agent increasing the endogenous synthesis of eicosapentaenoic acid (EPA) and of docosahexaenoic acid (DHA), for preparing a food, health diet or pharmaceutical composition intended to be administered to a human being or to an animal.

In an embodiment of the invention, the polyphenol may be selected from apigenin, luteolin, chlorogenic acid, caffeic acid, ferulic acid, cumaric acids, hydroxycinnamic acid, salicylic acid, gallic acid, quercetin, eriodyctiol, catechin, epicatechin, epigallocatechin gallate, isorhamnetin, kaempherol, hesperetin, naringenin, phloretin, enterolactone, enterodiol, oleuropein, hydroxytyrosol, resveratrol, myricetin, rutin, phenolic acids, stilbenes, flavonoids and lignans.

Preferably, the polyphenol is an anthocyanin.

In a quite advantageous way, the anthocyanin is selected from cyanidin, delphidin, mavidin, peonidin, pelargonidin and petunidin.

In an embodiment of the invention, the composition further comprises a compound selected from vitamins, mineral salts and trace elements.

Advantageously, the composition is found in the form of granules, powder, liquid, semi-liquid.

Preferably, when the composition is a food or health diet composition, it may be an oil, a margarine, a yoghurt, a cheese, a bread, a rusk, a cake, a biscuit, a meal substitute, a cream, a chocolate bar, a cereal bar or further a fruit-based compote.

In the sense of the present invention, by “a food or health diet composition” is meant any type of product intended to be ingested by animal, notably human, organisms. Food supplements notably enter the field of protection of the present invention. Food supplements are products intended to be ingested, as a supplement to current food, in order to compensate for insufficiency of daily intakes of certain compounds. The food or health diet composition of the invention may be in the form of granules, powder, in liquid form naturally or suspended or put into a solution. It may appear in a suitable form for addition to the food ration of an animal or to any other product forming a food supplement. As such, the composition according to the invention may be in a dry, pasty, semi-pasty liquid or semi-liquid form. For example, these may be food products, beverages, food supplements and nutraceutical products.

Among the food products intended for human beings, more particularly relevant to the present invention, mention may be made of oils, margarines and other fats, yoghurts, cheeses, notably fresh cheeses and derived products, fermented products, dairy products, bread, rusks, and other cereal products or derived therefrom (for example pasta), cakes and biscuits, meal substitutes, snacks in general, foods intended for children, babies and infants, creams, desserts, ice creams, chocolate bars, cereal bars, fruit-based compotes.

According to an embodiment and in accordance with the present invention, the composition is in a suitable form for addition to the food ration of an animal. By “animal”, is more particularly meant in addition to humans, livestock and notably grazing animals (notably cattle reared for meat, milk and other dairy products, cheese and leather; sheep reared for meat, wool and cheese; goats; pigs), rabbits, poultry (chickens, hens, turkeys, ducks, geese and other poultry) reared for their meats and derived products including eggs, aquatic animals (for example animals from marine farms, fish, shrimps, oysters and mussels), leisure animals and pets (notably horses, dogs, cats, pet birds, aquarium fish), laboratory animals (notably rats and mice).

By “pharmaceutical composition” is notably but not exclusively meant compositions in solid, liquid, pasty, semi-pasty, semi-liquid form. When the present invention relates to a pharmaceutical composition, it contains a pharmaceutically acceptable excipient. The latter is selected so as to be suitable for formulating the two compounds of the composition (fatty acid and polyphenol). The excipient is adapted to the desired administration route and to the nature of the desired dosage form. The pharmaceutical compositions of the invention are available in any dosage forms suitable for administration. Said dosage forms may notably consist in: tablets, gelatin capsules, powders, granules, lyophilizates, drinkable solutes, syrups, suspensions and suppositories. This list is not exhaustive. The term of “tablet” designates any kinds of tablets and notably effervescent tablets, dispersible tablets and orodispersible tablets.

When the composition of the invention is in the form of a granule or tablet, it may be in a coated form in order to avoid enzymatic destruction which occurs at a certain pH, and at the same time so as to allow controlled release of the active compound in another portion of the digestive tract. The composition according to the invention may also be found as sustained release or controlled release tablets.

By “a fatty acid of the omega-3 group”, is meant a polyunsaturated fatty acid, i.e. having at least one double bond, and for which the first double bond binds the carbon atoms 3 and 4. As an indication, the fatty acid may include 3, 4 or 5 double bonds. The fatty acid of the omega-3 group may include 18, 20, 22 or 24 carbon atoms. For example, the fatty acid of the omega-3 group may be an α-linolenic acid (ALA;C18:3), a stearidonic acid (SA:C18:4), an eicosatetraenoic acid (ETA;C20:4), an eicosapentaenoic acid (EPA;C20:5), a docosapentaenoic acid (DPA; C22:5), or a mixture of at least two of these compounds.

According to an embodiment of the present invention, the fatty acid of the omega-3 group is α-linolenic (ALA;C18:3) or stearidonic acid (SA:C18:4).

Polyphenols form a family of organic molecules characterized by the presence of several phenol groups. Their structure is more or less complex.

According to the present invention, the polyphenols are selected from those generally present in so-called Mediterranean foodstuffs, for example grapes, wine, citrus fruit, full cereals, legumes (notably lentils, peas, broad beans), teas, coffee, olive oil, olives, flax seeds, flax oil, walnuts, walnut oil, fruit (notably cherries, plums, raspberries, blueberries, strawberries, gooseberries, blackberries, cranberries) and vegetables (notably cucumbers, knob celery, zucchinis, cruciferous vegetables such as broccoli, artichokes, garlic, egg-plants, tomatoes, red onions, pimentos, carrots), and aromatic herbs (notably thyme, basil, parsley, coriander, oregano, rosemary, chives, sage). According to the present invention, the polyphenols are also selected from those generally present in foodstuffs containing fats, for example the polyphenols of cocoa, grape seed, Aztec corn.

According to an embodiment of the present invention, the polyphenols are selected from apigenin, luteolin, chlorogenic acid, caffeic acid, ferulic acid, cumaric acids, hydroxycinnamic acid, salicylic acid, gallic acid, quercetin, eriodyctiol, catechin, epicatechin, epigallocatechin gallate, isorhamnetin, kaempherol, hesperetin, naringenin, phloretin, enterolactone, enterodiol, oleuropein, hydroxytyrosol, resveratrol, myricetin and rutin.

According to another embodiment of the present invention, the polyphenols are selected from phenolic acids, stilbenes, flavonoids and lignans. When the polyphenols are flavonoids, they are selected from anthocyanins and proanthocyanidins, flavonols, flavones, flavanones, flavanols, isoflavones and chalcones. Examples of anthocyanins are cyanidin, delphidin, malvidin, peonidin, pelargonidin and petunidin.

According to an embodiment of the present invention, the composition further comprises a compound selected from vitamins, mineral salts and trace elements.

Among vitamins, mention may be made of vitamin C (ascorbic acid), vitamin B3 (nicotinamide), vitamin B5 (pantothenic acid), vitamin B6 (pyridoxine), vitamin B2 (riboflavin), vitamin B1, (thiamine), vitamin B9 (folic acid), vitamin B8 (biotin), vitamin B 12 (cyanocobalamine), vitamin D (calciferol), vitamin E (tocopherol), vitamin A (retinol) and vitamin K.

Among mineral salts, mention may be made of calcium (Ca), iron (Fe), magnesium (Mg), sodium (Na), phosphorous (P), potassium (K), sulphur (S).

Among the trace elements, mention may be made of aluminium (Al), arsenic (As), boron (B), chlorine (Cl), chromium (Cr), cobalt (Co), copper (Cu), fluorine (F), iodine (I), manganese (Mn), molybdenum (Mo), nickel (Ni), lead (Pb), silicone (Si), selenium (Se), vanadium (V) and zinc (Zn).

The compositions of the present invention are prepared by mixing of said compounds.

The food, health diet or pharmaceutical composition comprises a) from 1 to 99% by weight of at least one fatty acid from the omega-3 group selected from α-linolenic acid and stearidonic acid; and b) from 99 to 1% by weight of said at least one polyphenol.

According to another embodiment, said at least one fatty acid from the omega-3 group selected from α-linolenic acid and stearidonic acid is present in an amount between 10 and 90%, preferably between 20 and 80% and even better between 30 and 70% by weight of the total weight of the composition.

According to another embodiment, said at least one polyphenol is present in an amount between 10 and 90%, preferentially between 20 and 80%, even better between 30 and 70% by weight of the total weight of the composition.

### EXAMPLE

### Equipment and Methods

The foodstuffs are prepared according to the procedure described in Toufektsian et al. (J. Nutr. 2008 ; 1 38 : 747-752). Each diet is equivalent in terms of energy to 60% carbohydrates, 23% proteins and 10% lipids. The composition of fatty acids of each diet is given in Table 1. The anthocyanins used in the present test come from transformed (not genetically) maize so as to make it rich in anthocyanins. In the case of the anthocyanin-rich diet, the food ration of the rats contains 20% of transformed maize, this percentage being relative to the total weight of maize present in the foodstuff (as pellets).

| Fatty acids | mg/100 g |
|---|---|
| C14:0 | 20 |
| C16:0 | 520 |
| C18:0 | 50 |
| C20:0 | 10 |
| C18:1n-9 | 595 |
| C16:1n-7 | 20 |
| C18:2n-6 | 1310 |
| C20:4n-6 | 3 |
| C22:4n-6 | — |
| C18:3n-3 | 110 |
| C20:5n-3 | 20 |
| C22:5n-3 | 10 |
| C22:6n-3 | 30 |
| Total SFA | 620 |
| Total MUFA | 510 |
| Total PUFA | 1480 |
| Total n-3 | 170 |
| Total n-6 | 1310 |

SFA: saturated fatty acids
PUFA: polyunsaturated fatty acids
MUFA: monounsaturated fatty acids

Three different procedures were followed.

### Procedure 1:

The study is conducted over 8 weeks. 31 animals are separated into 2 experimental groups. The animals of the first group (N=15) are fed with a base diet, without any anthocyanin (ACN-free). The animals of the second group (N=16) are fed with the same base diet to which anthocyanins are added(ACN-rich).

### Procedure 2:

The study is conducted over 8 weeks. 25 animals are separated into two experimental groups. The animals of the first group (N=15) are fed with a base diet without any anthocyanin (ACN-free). The animals of the second group (N=10) are fed with a base diet with anthocyanins (ACN rich). The animals of both groups further receive a daily oral administration of palm oil (80 μl/d). This complementation provides 37 mg/d of SFA, 28 mg/d of MUFA and 7 μg/d of PUFA. The palm oil does not contain any ALA, or EPA or DHA.

### Procedure 3:

The study is conducted over 8 weeks. 29 animals are separated into two experimental groups. The animals of the first group (N=18) are fed with a base diet without any anthocyanin (ACN-free). The animals of the second group (N=11) are fed with a base diet with anthocyanins (ACN rich). The animals of both groups further receive a daily oral administration of polyunsaturated acids (N−3 with long chains: EPA and DHA at 60 mg/d in the form of ethyl esters), not containing any ALA.
In the three procedures above, the body mass and the food consumption are measured each week. At the end of the experimental period (8 weeks) the animals are anaesthetized and heparinized. After excision of the heart, the blood is connected in the thorax. The plasma is obtained after centrifugation (5 minutes, 1,000G, 4° C.). The samples are kept at 80° C. for the subsequent fatty acid analyses.

### Results

### Body Mass and Food Consumption

Procedure 1:

The time-dependant change in the body mass is similar both in ACN-free rats and ACN-rich rats. The daily food consumption is equivalent in both groups (ACN-free: 22.0±0.4 g vs. ACN-rich: 21.8±0.3 g, P=NS).

Procedure 2:

In rats supplemented with palm oil, the body mass and the daily food intake (ACN-free plus palm oil: 23.4±0.8 g vs. ACN-rich plus palm oil: 23.3±1.1 g, P=NS) are not significantly affected by the consumption of anthocyanins.

Procedure 3:

Similarly, food anthocyanins neither induce modifications in the weight gain nor in the daily food consumption (ACN-free±n-3: 21.8±0.6 g vs. ACN-rich±n-3: 22.0±0.3 g, P=NS) in rats further receiving an oral daily supplement of polyunsaturated acids.

### Effect of Anthocyanins on the Metabolism of Fatty Acids

### See Tables 2, 3 and 4.

Procedure 1:

As shown in Table 2, the level of triglycerides and cholesterol are not significantly modified by the consumption of anthocyanin. In rats supplemented with polyphenol (ACN-rich) the plasma levels of oleic, myristic and palmitic fatty acids are similar to those of rats not supplemented with polyphenol (ACN-free). The levels of SFA, MUFA, PUFA and n-6 (or omega-6) are similar in both groups. The n-3 (or omega-3) levels and the n-3:n-6 ratio are nevertheless significantly increased after consumption of polyphenol (ACN-rich). In this respect, the EPA and DHA levels are increased by 41% and 16%, respectively.

Procedure 2:

As shown in Table 3, the SFA, MUFA and PUFA levels are comparable in both groups. The n-3 and n-6 levels are not significantly different. However, the n-3:n-6 ratio significantly increases in animals receiving together the palm oil+anthocyanins combination. In this respect, the EPA and DHA levels are increased by 25% and 10%, respectively.

Procedure 3:

As shown in Table 4, the plasma levels of SFA, MUFA, PUFA and n-6 are similar in both groups. On the other hand, the n-3 levels and the n-3:n-6 ratio are increased in animals receiving together the EPA+DHA+anthocyanins combination. In this respect the EPA levels on the one hand and the DHA levels on the other hand are increased by 24% and 16% respectively.

TABLE 2

| | |
|---|---|
| ACN-free | ACN-rich |
| (n = 15) | (n = 16) |

Blood lipids (g/L)

| | | |
|---|---|---|
| Total Cholesterol | 0.41 ± 0.07 | 0.5 ± 0.02 |
| HDL Cholesterol | 0.37 ± 0.02 | 0.39 ± 0.02 |

Fatty acids (% of total fatty acids)

| | | | |
|---|---|---|---|
| Myristic | 14:0 | 0.77 ± 0.07 | 0.72 ± 0.08 |
| Palmitic | 16:0 | 21.94 ± 0.65 | 20.57 ± 0.68 |
| Stearic | 18:0 | 6.30 ± 0.39 | 6.02 ± 0.37 |
| Oleic | 18:1n-9 | 12.84 ± 0.74 | 11.93 ± 1.13 |
| Linolenic (LA) | 18:2n-6 | 23.41 ± 0.92 | 23.81 ± 0.51 |
| α-linolenic (ALA) | 18:3n-3 | 0.75 ± 0.08 | 0.76 ± 0.07 |
| Arachidonic(AA) | 20:4n-6 | 16.14 ± 1.58 | 17.53 ± 1.75 |
| Eicosapentaenoic (EPA) | 20:5n-3 | 0.86 ± 0.03 | 1.21 ± 0.09** |
| Docosapentaenoic (DPA) | 22:5n-3 | 0.63 ± 0.05 | 0.74 ± 0.05 |
| Docosahexaenoic (DHA) | 22:6n-3 | 3.85 ± 0.22 | 4.48 ± 0.21* |
| Total SFA | | 29.07 ± 0.63 | 27.37 ± 0.60 |
| Total MUFA | | 23.49 ± 1.41 | 22.43 ± 1.84 |
| Total PUFA | | 47.25 ± 1.56 | 50.03 ± 2.12 |
| Total n-3 | | 6.17 ± 0.24 | 7.21 ± 0.27** |
| Total n-6 | | 41.08 ± 1.38 | 42.82 ± 1.98 |
| n-3:n-6 ratio | | 0.15 ± 0.00 | 0.17 ± 0.01** |

*P < 0.05,
**P < 0.01 vs. ACN-free)
SFA: saturated fatty acids
PUFA: polyunsaturated fatty acids
MUFA: monounsaturated fatty acids

TABLE 3

| | |
|---|---|
| ACN-free | ACN-rich |
| (n = 15) | (n = 10) |

Blood lipids (g/L)

| | | |
|---|---|---|
| Total Cholesterol | 0.39 ± 0.02 | 0.42 ± 0.02 |
| HDL Cholesterol | 0.32 ± 0.01 | 0.35 ± 0.02 |

Fatty acids (% of total fatty acids)

| | | | |
|---|---|---|---|
| Myristic | 14:0 | 1.03 ± 0.08 | 1.09 ± 0.08 |
| Palmitic | 16:0 | 22.65 ± 0.27 | 22.96 ± 0.56 |
| Stearic | 18:0 | 7.73 ± 0.45 | 8.00 ± 0.67 |
| Oleic | 18:1n-9 | 11.29 ± 0.75 | 11.18 ± 0.61 |
| Linolenic (LA) | 18:2n-6 | 20.29 ± 0.42 | 19.75 ± 0.77 |
| α-linolenic (ALA) | 18:3n-3 | 0.48 ± 0.06 | 0.36 ± 0.03 |
| Arachidonic (AA) | 20:4n-6 | 18.83 ± 1.11 | 17.05 ± 1.08 |
| Eicosapentaenoic | 20:5n-3 | 1.31 ± 0.07 | 1.64 ± 0.09* |
| Docosapentaenoic | 22:5n-3 | 0.90 ± 0.05 | 0.87 ± 0.05 |
| (DPA) | | | |
| Docosahexaenoic | 22:6n-3 | 4.93 ± 0.13 | 5.44 ± 0.25* |
| (DHA) | | | |
| Total SFA | | 31.43 ± 0.57 | 32.04 ± 0.88 |
| Total MUFA | | 19.86 ± 1.07 | 20.48 ± 1.31 |
| Total PUFA | | 48.52 ± 1.15 | 46.82 ± 1.64 |
| Total n-3 | | 7.62 ± 0.25 | 8.32 ± 0.36 |
| Total n-6 | | 40.90 ± 1.14 | 38.50 ± 1.43 |
| n-3:n-6 ratio | | 0.19 ± 0.01 | 0.22 ± 0.01* |

(*P < 0.05 vs. ACN-free + palm oil)
SFA: saturated fatty acids
PUFA: polyunsaturated fatty acids
MUFA: monounsaturated fatty acids

TABLE 4

| | |
|---|---|
| ACN-free | ACN-rich |
| (n = 18) | (n = 11) |

Blood lipids (g/L)

| | | |
|---|---|---|
| Total Cholesterol | 0.37 ± 0.01 | 0.35 ± 0.02 |
| HDL Cholesterol | 0.31 ± 0.01 | 0.28 ± 0.02 |

Fatty acid (% of total fatty acids)

| | | | |
|---|---|---|---|
| Myristic | 14:0 | 0.99 ± 0.05 | 0.82 ± 0.08 |
| Palmitic | 16:0 | 22.39 ± 0.26 | 21.91 ± 0.73 |
| Stearic | 18:0 | 7.95 ± 0.36 | 7.94 ± 0.52 |
| Oleic | 18:1n-9 | 9.35 ± 0.34 | 10.07 ± 0.44 |
| Linolenic (LA) | 18:2n-6 | 21.96 ± 0.63 | 20.85 ± 0.76 |
| α-linolenic (ALA) | 18:3n-3 | 0.38 ± 0.03 | 0.43 ± 0.03 |
| Arachidonic (AA) | 20:4n-6 | 14.45 ± 0.50 | 12.86 ± 0.88 |
| Eicosapentaenoic (EPA) | 20:5n-3 | 4.11 ± 0.18 | 09 ± 0.38* |
| Docosapentaenoic (DPA) | 22:5n-3 | 1.36 ± 0.06 | 1.56 ± 0.08* |
| Docosahexaenoic (DHA) | 22:6n-3 | 7.74 ± 0.31 | 9.00 ± 0.30** |
| Total SFA | | 31.33 ± 0.48 | 30.68 ± 1.01 |
| Total MUFA | | 16.83 ± 0.62 | 17.68 ± 0.99 |
| Total PUFA | | 51.64 ± 1.00 | 51.47 ± 1.60 |
| Total n-3 | | 13.60 ± 0.46 | 16.07 ± 0.60** |
| Total n-6 | | 38.04 ± 0.94 | 35.39 ± 1.59 |
| n-3:n-6 ratio | | 0.36 ± 0.02 | 0.47 ± 0.36** |

(*P < 0.05, **P < 0.01 vs. ACN-free + n-3)
SFA: saturated fatty acids
PUFA: polyunsaturated fatty acids
MUFA: monounsaturated fatty acids

### CONCLUSION

A notable increase in EPA and EHA is observed. The anthocyanins have a clear effect on omega-3s, but no significant effect on omega-6s. The only difference between both groups of rats being the anthocyanin intakes, it is these substances to which the effect on omega-3s should be ascribed. It should be noted that in these experiments, the ALA provisions were identical in the groups either supplemented with anthocyanins or not.

1. The use of at least one fatty acid of the omega-3 group selected from α-linolenic acid and stearidonic acid and of at least one polyphenol, as an agent increasing endogenous synthesis of eicosapentaenoic acid and docosahexaenoic acid, for the preparation of a food, health or pharmaceutical composition intended to be administered to a human being or to an animal.

2. The use according to claim 1, characterizing that the polyphenol is selected from apigenin, luteolin, chlorogenic acid, caffeic acid, ferulic acid, cumaric acid, hydroxycinnamic acid, salicylic acid, gallic acid, quercetin, eriodyctiol, catechin, epicatechin, epigallocatechin gallate, isorhamnetin, kaempherol, hesperetin, maringenin, phloretin, enterolactone, enterodiol, oleuropein, hydroxytyrosol, resveratrol, myricetin, rutin, phenolic acids, stilbenes, flavonoids and lignans.

3. The use according to claim 1, characterizing that said polyphenol is an anthocyanin.

4. The use according to claim 3, characterizing that said anthocyanin is selected from cyanidin, delphidin, malvidin, peonidin, pelargonidin and petunidin.

5. The use according to claim 1, characterizing that said composition further comprises a compound selected from vitamins, mineral salts and trace elements.

6. The use according to claim 1, characterizing that said composition is in the form of granules, powder, liquid, semi-liquid.

7. The use according to claim 1, characterizing that the food or health diet composition is an oil, a margarine, a yoghurt, a cheese, a bread, a rusk, a cake, a biscuit, a meal substitute, a cream, a chocolate bar, a cereal bar, a fruit-based compote.
